SEQUENCE LISTING (7 sequences)

SEQ ID NO: 1 - DNA, 49 bp, synthetic construct
  source 1..49: mol_type = other DNA
tactcgttatttcgtagcacttttccccaccaccttggtgttttttttt

SEQ ID NO: 2 - DNA, 39 bp, synthetic construct
  source 1..39: mol_type = other DNA
ctcccaaccgctccaccctgcctccgcctcttttttttt

SEQ ID NO: 3 - DNA, 33 bp, synthetic construct
  source 1..33: mol_type = other DNA
caaaagtgcacgctactttgctaattttttttt

SEQ ID NO: 4 - DNA, 40 bp, synthetic construct
  source 1..40: mol_type = other DNA
tggggggtggttgggggtagtatatcgggtcagtggtgcg

SEQ ID NO: 5 - DNA, 30 bp, synthetic construct
  source 1..30: mol_type = other DNA
ccccccagtccgtcctcccagcctcacacc

SEQ ID NO: 6 - DNA, 49 bp, synthetic construct
  source 1..49: mol_type = other DNA
gttgggcacgtgttgtctctctgtgtctcgtgcccttcgctaggcccac

SEQ ID NO: 7 - skipped

(19) United States
(12) Patent Application Publication
(10) Pub. No.: US 2025/0283878 A1
(43) Pub. Date: Sep. 11, 2025

(54) NUCLEIC ACID APTAMER TEST STRIP, PREPARATION METHOD THEREFOR, AND USE THEREOF

(71) Applicant: QINGDAO AGRICULTURAL UNIVERSITY, Qingdao (CN)

(72) Inventors: Wei WU, Qingdao (CN); Yiping XU, Qingdao (CN); Jie ZHAO, Qingdao (CN); Qinzhi WANG, Qingdao (CN); Juan TANG, Qingdao (CN); Xiudan HOU, Qingdao (CN); Qingli YANG, Qingdao (CN); Yongchao MA, Qingdao (CN); Liangtao LV, Qingdao (CN)

(21) Appl. No.: 19/183,799

(22) Filed: Apr. 19, 2025

Related U.S. Application Data
(63) Continuation of application No. PCT/CN2023/124890, filed on Oct. 17, 2023.

(30) Foreign Application Priority Data
Oct. 21, 2022 (CN) 202211292617.6

(51) Int. Cl. G01N 33/569; C12N 15/115; G01N 33/543; G01N 33/553

(52) U.S. Cl. CPC G01N 33/569; C12N 15/115; G01N 33/54346; G01N 33/553; C12N 2310/16; G01N 2333/245; G01N 2333/31

(57) ABSTRACT

The present invention discloses nucleic acid aptamer test strip, preparation method and application thereof, which belongs to technical field of pathogen detection. The nucleic acid aptamer test strip includes sample pad, binding pad, nitrocellulose membrane, absorbent pad and backing plate. The binding pad contains one or more capture aptamer. The nitrocellulose membrane contains one or several detection lines and one quality control line, wherein detection line contains detection aptamer, and wherein quality control line contains quality control aptamer. The capture aptamer on binding pad is modified on to silver-core gold-shell nanoparticles. The nucleic acid aptamer test strip of the present invention improves detection sensitivity, reduces detection costs, is simple to operate, can be detected and observed with naked eye, has high stability, and is suitable for onsite rapid detection.

This application is a Continuation application of PCT/CN2023/124890, filed on Oct. 17, 2023, which claims priority to Chinese Patent Application No. CN 202211292617.6, filed on Oct. 21, 2022, which is incorporated by reference for all purposes as if fully set forth herein.

A Sequence Listing XML file named “10037_0042.xml” created on Apr. 19, 2025 and having a size of 6,638 bytes, is filed concurrently with the specification. The sequence listing contained in the XML file is part of the specification and is herein incorporated by reference in its entirety.

### TECHNICAL FIELD

The present invention relates to the technical field of pathogen detection, specifically to a nucleic acid aptamer test strip, preparation method and application thereof.

### TECHNICAL BACKGROUND

Listeria monocytogenes is a zoonotic pathogen that is generally transmitted through food. After infection, the main symptoms are sepsis, meningitis and monocytosis. It is widely present in nature and can still grow and reproduce in an environment of 4° C. It is one of the main pathogens in the refrigerated food that threaten human health. Staphylococcus aureus (S. aureus) is a common facultative anaerobic Gram-positive pathogen that produces a variety of toxins as well as enzymes. It is capable of producing multiple toxins and enzymes, leading to pyogenic infections in humans and animals, as well as food-borne illnesses. It can contaminate food during processing, packaging, and transportation. The toxins secreted by it are not sensitive to heat and can still cause the illness even if it is heated and boiled for 30 minutes. Escherichia coli O157:H7 (E. coli) is common cause of food-borne illness outbreaks around the world. It is widely distributed in nature and can cause gastrointestinal infections in humans or animals, resulting in severe abdominal cramping pain and recurrent episodes of hemorrhagic diarrhea, accompanied by fever, vomiting and other symptoms. Foods of animal origin such as beef, chicken, milk, dairy products, etc. are main factors responsible for transmission of Escherichia coli through food.

At present, the main detection methods for food-borne pathogen include traditional culture method, molecular detection method, immunodetection method as well as biosensor method. The traditional culture method takes a long time to detect and its detection process is complicated. The molecular detection method such as common PCR detection method has high detection sensitivity and can achieve automated detection, but relies on the expensive equipment. The immunodetection method, such as enzyme-linked immunosorbent assay (ELISA), enables specific detection of food-borne pathogen through antigen-antibody interactions. However, they are susceptible to the false-positive results. The biosensor method uses sensors and other analytical equipment to achieve the rapid as well as portable detection of food-borne pathogen. The biosensor detection is an emerging technology in recent years that can achieve the highly sensitive and rapid detection of food-borne pathogen, but its detection relies on corresponding equipment as well and is therefore not portable enough. Based on these, it is of the great significance to provide a detection system that is portable, efficient, stable, observable by the naked eye, and can achieve simultaneous detection of the above three pathogens.

### SUMMARY OF THE INVENTION

The present invention provides a nucleic acid aptamer test strip, comprising a sample pad, a binding pad, a nitrocellulose membrane, a absorbent pad and a backing plate, wherein the binding pad contains one or more capture aptamers, wherein the nitrocellulose membrane contains one or more detection lines and one quality control line, wherein detection line contains detection aptamer, wherein the quality control line contains quality control aptamer, and wherein the capture aptamer on the binding pad is modified onto silver-core gold-shell nanoparticles.

In the above-mentioned nucleic acid aptamer test strip, capture aptamer is used to capture pathogen. The detection aptamer can form a sandwich complex with capture aptamer that captures pathogen, so that capture aptamer that captures pathogen is retained on detection line. The quality control aptamer is complementary chain of capture aptamer, which can bind to the empty capture aptamer through complementary action, so that empty capture aptamer can be retained on quality control line.

The present invention provides application of above-mentioned nucleic acid aptamer test strip in detection of pathogen for non-diagnostic purposes. For example, nucleic acid aptamer test strip is applied to pathogen detection in food. The food includes but is not limited to milk, cheese, cooked meat products, beef, cold meats and leftovers that are not thoroughly heated. The above-mentioned pathogens are preferably selected from food-borne pathogens, including but not limited to one or more of Listeria monocytogenes, Staphylococcus aureus and Escherichia coli O157:H7.

In the above-mentioned nucleic acid aptamer test strip, preferably:

The capture aptamer is selected from the nucleic acid aptamer A1, nucleic acid aptamer A2 or nucleic acid aptamer A3. The detection aptamer is selected from nucleic acid aptamer B1, nucleic acid aptamer B2 or nucleic acid aptamer B3. The quality control aptamer is selected from nucleic acid aptamer C. Among them, sequence of nucleic acid aptamer A1 is shown in SEQ ID NO:1, sequence of nucleic acid aptamer A2 is shown in SEQ ID NO:2, sequence of nucleic acid aptamer A3 is shown in SEQ ID NO:3, sequence of nucleic acid aptamer B1 is shown in SEQ ID NO:4, sequence of nucleic acid aptamer B2 is shown in SEQ ID NO:5, sequence of nucleic acid aptamer B3 is shown in SEQ ID NO:6, and sequence of nucleic acid aptamer C is shown in SEQ ID NO:7.

In one specific embodiment, nucleic acid aptamer test strip of present invention comprises the sample pad, the binding pad, the nitrocellulose membrane, the absorbent pad and the backing plate. The binding pad contains nucleic acid aptamer A1. The nitrocellulose membrane contains one detection line as well as one quality control line, wherein the detection line contains nucleic acid aptamer B1, and wherein the quality control line contains nucleic acid aptamer C. The nucleic acid aptamer test strip can be used for the detection of Listeria monocytogenes for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A2. The nitrocellulose membrane contains one detection line as well as one quality control line, wherein the detection line contains nucleic acid aptamer B2, and wherein the quality control line contains nucleic acid aptamer C. The nucleic acid aptamer test strip can be used for the detection of Staphylococcus aureus for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A3. The nitrocellulose membrane contains one detection line as well as one quality control line, wherein the detection line contains the nucleic acid aptamer B3, and wherein the quality control line contains nucleic acid aptamer C. The nucleic acid aptamer test strip can be used for the detection of Escherichia coli O157:H7 for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A1 as well as nucleic acid aptamer A2. The nitrocellulose membrane contains two detection lines as well as one quality control line, wherein one detection line contains nucleic acid aptamer B1, wherein other detection line contains nucleic acid aptamer B2, and wherein the quality control line contains nucleic acid aptamer C. The nucleic acid aptamer test strip can be used for the detection of Listeria monocytogenes and Staphylococcus aureus for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A1 as well as nucleic acid aptamer A3. The nitrocellulose membrane contains two detection lines as well as one quality control line, wherein one detection line contains nucleic acid aptamer B1, wherein other detection line contains nucleic acid aptamer B3, and wherein the quality control line contains nucleic acid aptamer C. The nucleic acid aptamer test strip can be used for detection of Listeria monocytogenes and Escherichia coli O157:H7 for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A2 as well as nucleic acid aptamer A3. The nitrocellulose membrane contains two detection lines as well as one quality control line, wherein one detection line contains nucleic acid aptamer B2, wherein other detection line contains nucleic acid aptamer B3, and wherein the quality control line contains nucleic acid aptamer C. The nucleic acid aptamer test strip can be used for the detection of Staphylococcus aureus and Escherichia coli O157:H7 for non-diagnostic purposes.

In one specific embodiment, nucleic acid aptamer test strip of present invention comprises the sample pad, the binding pad, the nitrocellulose membrane, the absorbent pad and the backing plate. The binding pad contains nucleic acid aptamer A1, nucleic acid aptamer A2 and nucleic acid aptamer A3. The nitrocellulose membrane contains three detection lines and one quality control line, wherein one detection line contains nucleic acid aptamer B1, wherein the other two detection lines contain nucleic acid aptamer B2 as well as nucleic acid aptamer B3 respectively, and wherein the quality control line contains nucleic acid aptamer C. The nucleic acid aptamer test strip can be used for detection of Listeria monocytogenes, Staphylococcus aureus as well as Escherichia coli O157:H7 for non-diagnostic purposes.

The above-mentioned nucleic acid aptamer A1, nucleic acid aptamer A2 and nucleic acid aptamer A3 are all modified on to silver-core gold-shell nanoparticles.

The above detection aptamer can be modified with biotin as well as streptavidin according to actual conditions, and the above quality control aptamer can be modified with biotin according to actual conditions.

The present invention provides a method for preparing the above-mentioned nucleic acid aptamer test strip, comprising the following steps:
cutting glass cellulose membrane into strip, soaking the strip in Tris-HCl buffer solution containing NaCl, Tween-20, bovine serum albumin, sucrose and Triton X-100, and drying the strip to prepare sample pad and binding pad; spraying the silver-core gold-shell nanoparticles modified with capture aptamer on binding pad, and drying strip for later use; spraying detection aptamer on nitrocellulose membrane to form the detection line; drawing line on the side of the quality control aptamer close to the absorbent pad to form the quality control line; drying sprayed nitrocellulose membrane for later use; sequentially adhering nitrocellulose membrane, sample pad, binding pad and absorbent pad to lining plate with an overlap of 2 mm between two adjacent pads, cutting the test strip into 4 mm wide strip after assembly, and storing the test strip in a dry place away from light.

The present invention provides a method for modifying the capture aptamer on the silver-core gold-shell nanoparticles, comprising the following steps:
adding capture aptamer to the TCEP solution for activation; placing the activated capture aptamer in the silver-core gold-shell nanoparticles solution for coupling reaction; dropping NaCl solution into reaction system, mixing thoroughly, and aging at the room temperature; centrifuging the reaction solution, discarding the supernatant, and then washing to obtain silver-core gold-shell nanoparticles modified with the capture aptamer after the reaction is completed.

The above silver-core gold-shell nanoparticles can be prepared by following method:
heating water to boiling, then adding silver nitrate solution and trisodium citrate solution in sequence, then stirring and reacting to generate the silver nanoparticles; adding hydroxylamine hydrochloride solution as well as chloroauric acid solution to silver nanoparticle solution, stirring and reacting, and after reaction is completed, silver-core gold-shell nanoparticles are generated.

The beneficial effects of the present invention are as follows:

The present invention uses silver-core gold-shell nanoparticles as the signal amplification element to replace colloidal gold used in traditional test strip, and prepares a new type of nucleic acid aptamer test strip. Compared with the ordinary test strip using colloidal gold, silver-core gold-shell nanoparticles can further amplify Raman signals to obtain more sensitive signals, and have better characteristics of SERS enhancement than colloidal gold, and can be combined with Raman imaging spectrometer for the quantitative analysis. In addition, the nucleic acid aptamer test strip of present invention adopts nucleic acid aptamer of food-borne pathogen instead of antibody, and utilizes sandwich method to capture pathogen, thereby improving detection sensitivity, reducing detection costs, being simple to operate, enabling the naked eye detection and observation, having high stability, and being suitable for onsite rapid detection.

### DESCRIPTION OF DRAWINGS

FIG. 1 shows composition diagram of nucleic acid aptamer test strip, comprising sample pad 1, binding pad 2, nitrocellulose membrane 3, detection line 4, quality control line 5, absorbent pad 6 and backing plate 7.

FIG. 2 shows specificity evaluation of nucleic acid aptamer test strip.

FIG. 3 shows sensitivity evaluation of nucleic acid aptamer test strip, wherein FIG. 3A is detection standard curve of Listeria monocytogenes, wherein FIG. 3B is detection standard curve of Staphylococcus aureus, and wherein FIG. 3C is detection standard curve of Escherichia coli O157:H7.

FIG. 4 shows stability evaluation of nucleic acid aptamer test strip.

FIG. 5 shows comparison experiment of affinity between nucleic acid aptamer as well as antibody.

### DETAILED DESCRIPTION OF THE EMBODIMENTS

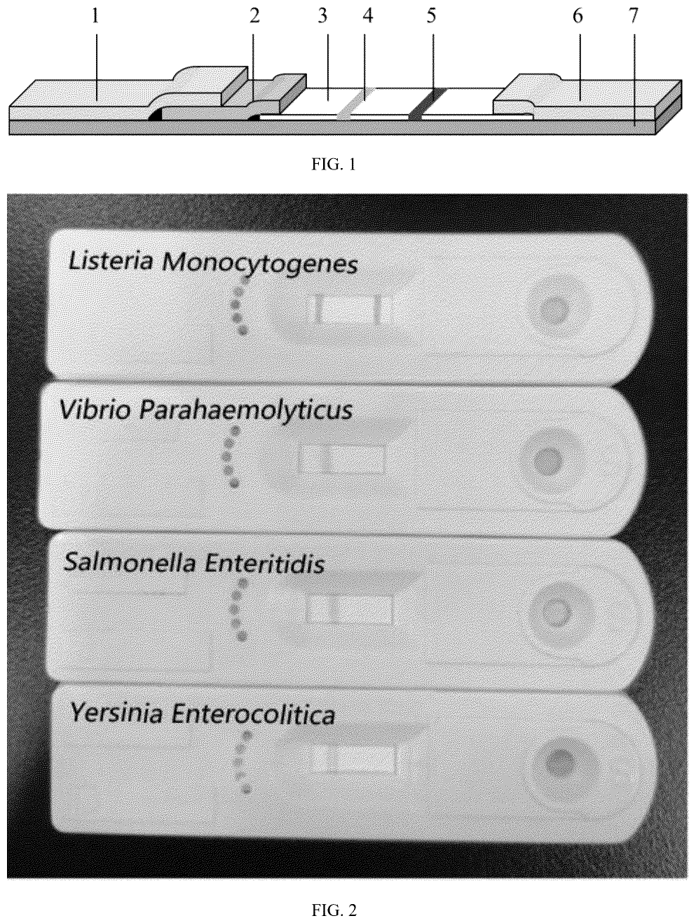

The present invention provides nucleic acid aptamer test strip, which comprises sample pad 1, binding pad 2, nitrocellulose membrane 3, absorbent pad 6 as well as backing plate 7. The nitrocellulose membrane 3, the sample pad 1, the binding pad 2 as well as the absorbent pad 6 are sequentially adhered to the backing plate 7 with an overlap of 2 mm between both adjacent pads. Among them, the nitrocellulose membrane 3 contains one detection line 4 and one quality control line 5, the detection line 4 is located on the side close to binding pad 2, and the quality control line 5 is located on the side close to absorbent pad 6, which is as shown in FIG. 1.

In the specific embodiment, one or more capture aptamers may be sprayed on the binding pad, wherein the capture aptamer on the binding pad are modified onto the silver-core gold-shell nanoparticles. One or a number of detection lines may be sprayed on the side of the nitrocellulose membrane close to the binding pad, and the detection lines contain detection aptamer. The quality control line may be sprayed on the side of the nitrocellulose membrane close to the absorbent pad, and the quality control line contains quality control aptamer.

The detection principle of the present invention is as follows:

The silver-core gold-shell nanoparticles themselves appear to be dark red. When sample pad contacts the solution containing pathogen, capture aptamer in the sample pad will specifically recognize and bind to pathogen and flow forward under push of liquid. When it flows to detection line, the detection aptamer forms a sandwich complex with capture aptamer that captures pathogen, resulting in the retention of capture aptamer on the detection line and producing a red color change. The capture aptamer that has not bound to pathogen (i.e., unloaded capture aptamer) is unable to form a sandwich complex with detection aptamer, allowing them to bypass the detection line and continue migrating. This aptamer subsequently binds to quality control aptamer on quality control line via complementary interactions, leading to its retention and causing the quality control line to turn red. When quality control line changes color, test result is credible. If quality control line does not change color, it means that the test strip is invalid and the test result is unreliable.

The present invention provides method for preparing silver-core gold-shell nanoparticles, comprising the following steps:

100 mL of ultrapure water was added to a 250 mL Erlenmeyer flask and heated to boiling under the stirring. Then, 1.6 mL of 0.1 M silver nitrate standard solution was added. After 1 minute, 3 mL of 1% trisodium citrate solution was added. The mixture was stirred and maintained at 100° C. for 30 minutes to synthesize silver nanoparticles. The resulting solution was stored at 4° C. Next, 40 mL of prepared silver nanoparticle solution was mixed with 1 mL of 5×10⁻³ M hydroxylamine hydrochloride solution and 4 mL of 4.65×10⁻⁴ M chloroauric acid solution. The mixture was stirred at room temperature for 1 hour to form silver-core gold-shell nanoparticles. The final product was stored at 4° C.

The present invention provides the method for modifying capture aptamer on silver-core gold-shell nanoparticles, comprising the following steps:

13 μL of 10 μM capture aptamer solution was mixed with 2 μL of 1 mM TCEP solution (tris(2-carboxyethyl)phosphine hydrochloride), and mixture was incubated at 4° C. for 2 hours for activation. 5 mL of silver-core gold-shell nanoparticle solution was centrifuged at 8,000 r/min for 20 minutes. The supernatant was discarded, and precipitate was redispersed in 5 mL of ultrapure water. The activated capture aptamer was then added, followed by the vortex mixing. The coupling reaction was carried out at the room temperature for 16 hours. After the reaction, 1% SDS solution was added to adjust final SDS concentration to 0.01%, and then the mixture was incubated at room temperature for 1 hour. Subsequently, 80 μL of 2 M NaCl solution was added drop wise in multiple times to achieve the final NaCl concentration of 160 mM. The mixture was thoroughly mixed and aged at 4° C. for 24 hours. The resulting solution was centrifuged at 12,000 r/min for 15 minutes. The supernatant was discarded, and pellet was washed once with ultrapure water to obtain silver-core gold-shell nanoparticles modified with capture aptamers. Silver-core gold-shell nanoparticles modified with the capture aptamer can be resuspended in 10 mM PBS suspension buffer (prepared by adding 2% sucrose, 0.5% Tween-20 and 0.25% BSA to 10 mM PBS), and stored at 4° C. in the dark place.

The present invention provides the following nucleic acid aptamers:

The capture aptamer is selected from aptamer A1, aptamer A2 or aptamer A3, wherein the aptamer A1 can be used to capture Listeria monocytogenes (ATCC19115), the aptamer A2 can be used to capture Staphylococcus aureus (ATCC6538), and the aptamer A3 can be used to capture Escherichia coli O157:H7 (ATCC8739). Detection aptamer is selected from aptamer B1, aptamer B2 or aptamer B3, wherein aptamer B1 can form a sandwich complex with aptamer A1 capturing Listeria monocytogenes, the aptamer B2 can form a sandwich complex with aptamer A2 capturing Staphylococcus aureus, and aptamer B3 can form a sandwich complex with aptamer A3 capturing Escherichia coli O157:H7. The quality control aptamer is selected from aptamer C.

The nucleic acid sequences of the above-mentioned nucleic acid aptamers are as follows:

Aptamer A1:
(SEQ ID NO: 1)
5′-TACTCGTTATTTCGTAGCACTTTTCCCCACCACCTTGGTGTTTTTT
TTTT-3′.
Aptamer A2:
(SEQ ID NO: 2)
5′-CTCCCAACCGCTCCACCCTGCCTCCGCCTTTTTTTTT-3′.
Aptamer A3:
(SEQ ID NO: 3)
5′-CAAAAGTGCACGCTACTTTGCTAATTTTTTTTT-3′.
Aptamer B1:
(SEQ ID NO: 4)
5′-TGGGGGGTGGTTGGGGGTAGTATATCGGGTCAGTGGTGCG-3′.
Aptamer B2:
(SEQ ID NO: 5)
5′-CCCCCCAGTCCGTCCTCCCAGCCTCACACC-3′.
Aptamer B3:
(SEQ ID NO: 6)
5′-GTTGGGCACGTGTTGTCTCTCTGTGTCTCGTGCCCTTCGCTAGGCC
CAC-3′
Aptamer C:
(SEQ ID NO: 7)
5′-AAAAAAAAA-3′.

Other terms used in the present invention, unless otherwise specified, generally have the meanings commonly understood by those of ordinary skill in the art. The present invention will be further described in detail below in conjunction with specific embodiments and with reference to data. The following embodiments are only intended to illustrate the present invention and are not intended to limit the scope of the present invention in any way.

### Embodiment 1

Preparation of Nucleic Acid Aptamer Test Strip with Single Detection Line:

### 1) Treatment of Glass Cellulose Membrane

The glass cellulose membrane was cut into strip, soaked in Tris-HCl buffer (0.1M, pH=8.0) containing 1% NaCl, 0.5% Tween-20, 1% bovine serum albumin, 2% sucrose and 1% Triton X-100 for 2 hours, and dried at 37° C. to prepare sample pad and binding pad. The silver-core gold-shell nanoparticles modified with capture aptamer were evenly sprayed on binding pad at 1 μL/cm using a gold sputter coater, and dried at 25° C. for later use.

The above capture aptamer is selected from the Listeria monocytogenes capture aptamer, the Staphylococcus aureus capture aptamer or the Escherichia coli O157:H7 capture aptamer, and the 5′ end is modified with a thiol group structure denoted as ‘HS-SH-C6’. In the present invention, the purpose of modifying the nucleic acid aptamer with ‘HS-SH-C6’ is to connect the aptamer to the nanoparticles through action of Au—S bond. The Au—S bond (gold-sulfur bond) is common self-assembly technology which mainly occurs between thiol groups as well as gold atoms to achieve chemical covalent bonding between nucleic acid aptamer and gold nanoparticles.

Listeria monocytogenes capture aptamer:
5′-HS SH C6-
TACTCGTTATTTCGTAGCACTTTTCCCCACCACCTTGGTGTTTTTTTTT
T-3′.
Staphylococcus aureus capture aptamer:
5′-HS SH C6-CTCCCAACCGCTCCACCCTGCCTCCGCCTTTTTTTT
T-3′.
Escherichia coli O157: H7 capture aptamer:
5′-HS SH C6-CAAAAGTGCACGCTACTTTGCTAATTTTTTTT-3′.

### 2) Preparation of Detection Line and Quality Control Line

The detection aptamer was streaked on the nitrocellulose membrane using a gold sputter coater to form detection line (Line T). The quality control aptamer was streaked on the side close to absorbent pad to form quality control line (Line C). The sprayed nitrocellulose membrane was dried at 25° C. for 2 h and stored in the dry environment at room temperature for later use.

The above detection aptamer is modified with biotin and streptavidin. The above quality control aptamer is modified with biotin. In the present invention, the detection aptamer is modified with biotin, and before spraying on the nitrocellulose membrane, streptavidin needs to be added to detection aptamer to allow biotin to react with streptavidin, so as to fix detection aptamer on the nitrocellulose membrane, so that it is not easily washed away by the liquid.

Streptavidin modification for the detection aptamer is as follows:

75 μL of 10 μM biotin-modified detection aptamer (manufactured by Sangon Biotech Co., Ltd.) was mixed with 25 μL of 1 mg/mL streptavidin solution and incubated at room temperature for 2 hours to allow for binding, resulting in the detection aptamer modified with both biotin and streptavidin. After reaction was complete, the mixture was centrifuged at 6,000 r/min at 4° C. for 20 minutes to remove unbound detection aptamer. The pellet was washed twice with 0.01 M PBS buffer, and the volume was adjusted back to the original using PBS buffer. The final product was stored at 4° C. for later use.

The detection aptamer is selected from the Listeria monocytogenes detection aptamer, the Staphylococcus aureus detection aptamer or Escherichia coli O157:H7 detection aptamer, which can respectively form sandwich complex with the Listeria monocytogenes detection aptamer, the Staphylococcus aureus detection aptamer or the Escherichia coli O157:H7 detection aptamer. The nucleic acid sequences after modification with biotin are as follows:

Listeria monocytogenes detection aptamer:
5′-biotin-TGGGGGGTGGTTGGGGGTAGTATATCGGGTCAGTGGTG
CG-3′.
Staphylococcus aureus detection aptamer:
5′-biotin-CCCCCCAGTCCGTCCTCCCAGCCTCACACC-3′.
Escherichia coli O157: H7 detection aptamer:
5′-biotin-
GTTGGGCACGTGTTGTCTCTCTGTGTCTCGTGCCCTTCGCTAGGCCCA
C-3′.

The above-mentioned quality control aptameric as follows:

5′-biotin-AAAAAAAAA-3′.

### 3) Assembly of Test Strip

The nitrocellulose membrane, sample pad, binding pad as well as the absorbent pad were adhered to backing plate in sequence with an overlap of 2 mm between two adjacent pads. After assembly, it was cut into 4 mm wide test strip and stored in a dry place away from light.

### 1. Pathogen Recovery Test

The spike recovery experiment was carried out using milk samples to measure bacterial suspensions of different concentrations. The bacterial suspensions of three food-borne pathogens (Staphylococcus aureus, Escherichia coli O157:H7 as well as Listeria monocytogenes) were added to the milk sample to make the final bacterial solution concentration reach addition concentration in Table 1. For example, final concentrations of Staphylococcus aureus were 1.14×10⁴ CFU/mL, 1.14×10⁴ CFU/mL and 1.14×10⁶ CFU/mL respectively. Milk containing different concentrations of bacterial solution was dripped onto test strip. After reaction was complete, the actual detection amount was determined by Raman spectrometer, and recovery rate was calculated. The feasibility of the detection experiment can be analyzed through the spike recovery experiment, simulating the situation of the detection method in the actual application. The higher the recovery rate, the smaller the error of the detection method and the higher the relative accuracy.

The test results are shown in Table 1:

TABLE 1
Detection recovery rate of pathogens at
different concentrations in milk samples

| | Addition Amount | Detection Amount | Recovery |
|---|---|---|---|
| Pathogen | (CFU/mL) | (CFU/mL) | Rate |
| Staphylococcus | 1.14 × 10⁴ | 1.01 × 10⁴ ± 241 | 88.6% |
| aureus | 1.14 × 10⁵ | 8.96 × 10⁴ ± 185 | 78.6% |
| | 1.14 × 10⁶ | 0.94 × 10⁶ ± 463 | 82.5% |
| Escherichia | 1.42 × 10⁴ | 1.09 × 10⁴ ± 311 | 76.8% |
| coli | 1.42 × 10⁵ | 1.17 × 10⁵ ± 114 | 82.4% |
| | 1.42 × 10⁶ | 1.23 × 10⁶ ± 263 | 86.6% |
| Listeria | 1.88 × 10⁴ | 1.56 × 10⁴ ± 150 | 83.0% |
| monocytogenes | 1.88 × 10⁵ | 1.34 × 10⁵ ± 362 | 71.3% |
| | 1.88 × 10⁶ | 1.37 × 10⁶ ± 537 | 72.9% |

As shown in Table 1, nucleic acid aptamer test strip of the present invention has a high recovery rate of 71.3% to 88.6% for Listeria monocytogenes, Staphylococcus aureus as well as Escherichia coli O157:H7. This shows that nucleic acid aptamer test strip of the present invention has a small error and a high accuracy rate in detecting food-borne pathogen.

### 2. Specificity Test

In order to avoid mutual influence of multiple pathogens, which may lead to large errors in the results, a single bacterial solution is used for the specificity test of test strip. Taking Listeria monocytogenes as an example, concentration of bacterial solution is 2×10⁶ CFU/mL, and Yersinia enterocolitica, Salmonella enteritidis and Vibrio parahaemolyticus at same concentration are used to verify the specificity of the test strip.

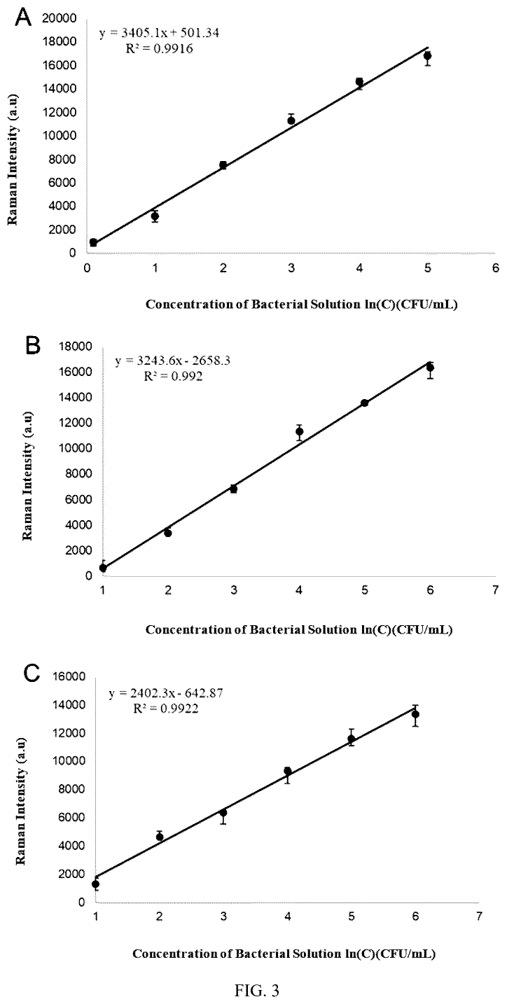

The test results are shown in FIG. 2:

The area of line C of the test strips all showed color reaction, and only the test strip for detecting Listeria monocytogenes showed a red strip on its line T, while line T on other test strips did not show color, which shows that nucleic acid aptamer test strip of the present invention has good specificity.

### 3. Sensitivity Test

Staphylococcus aureus, Escherichia coli as well as Listeria monocytogenes at the different concentrations were added to test strips, and after the reaction was complete, results were measured by Raman spectrometer and corresponding regression equation was calculated. The concentrations were set as: 1×10¹ CFU/mL, 1×10² CFU/mL, 1×10³ CFU/mL, 1×10⁴ CFU/mL, 1×10⁵ CFU/mL, 1×10⁶ CFU/mL

The test results are shown in FIG. 3:

FIG. 3 shows standard curves of three pathogens. The Raman intensity is proportional to target concentration in range of 10¹ to 10⁶ CFU/mL. The linear equation of Listeria monocytogenes is y=3405.1×+501.34, R²=0.9916 (FIG. 3A). The linear equation of Staphylococcus aureus is y=3243.6x-2658.3, R²=0.992 (FIG. 3B). The linear equation of Escherichia coli is y=2402.3x-642.87, R²=0.9922 (FIG. 3C). As concentration of bacterial solution increases, the color of line T deepens. Among them, the detection limit of Listeria monocytogenes by naked eye is 1.88×10³ CFU/mL, the detection limit of Staphylococcus aureus by naked eye is 1.14×10³ CFU/mL, and the detection limit of Escherichia coli O157:H7 by naked eye is 1.42×10³ CFU/mL.

### 4. Stability Test

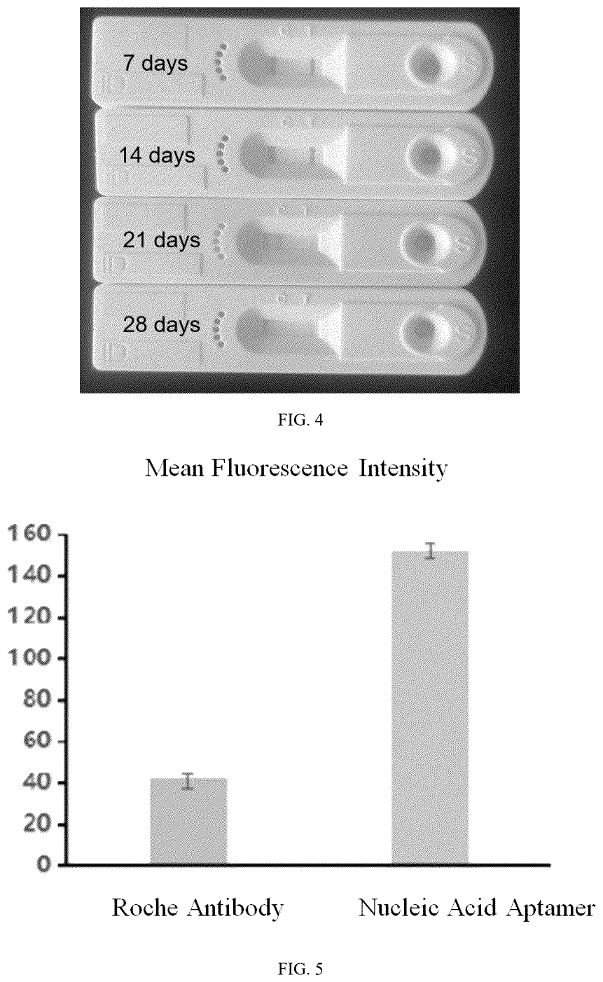

After nucleic acid aptamer test strip was placed for 7 days, 14 days, 21 days, and 28 days, the color development of line T was observed by taking Listeria monocytogenes as example, and the results are shown in FIG. 4. As can be seen from FIG. 4, after being placed for 7 days, 14 days as well as 21 days, the color development of line T of the test strip is still obvious, and the color development of the line T becomes weaker after 28 days. Therefore, the nucleic acid aptamer test strip of the present invention still maintains a clear and visible color within 21 days of the color development, and its stability is good.

### Embodiment 2

Preparation of Nucleic Acid Aptamer Test Strip with Multiple Detection Lines:

### 1) Treatment of Glass Cellulose Membrane

The glass cellulose membrane was cut into strip, soaked in Tris-HCl buffer (0.1 M, pH=8.0) containing 1% NaCl, 0.5% Tween-20, 1% bovine serum albumin, 2% sucrose and 1% Triton X-100 for 2 h, and dried at 37° C. to prepare sample pad and binding pad. The silver-core gold-shell nanoparticles modified with Listeria monocytogenes capture aptamer, Staphylococcus aureus capture aptamer and Escherichia coli O157:H7 capture aptamer respectively were evenly sprayed on the binding pad at 1 μL/cm using a gold sputter coater, and dried at 25° C. for later use.

### Preparation of Detection Line and Quality Control Line

The Listeria monocytogenes detection aptamer, Staphylococcus aureus detection aptamer and Escherichia coli O157:H7 detection aptamer were streaked on nitrocellulose membrane using a gold sputter coater to form three detection lines (line T). The quality control aptamer was streaked on the side close to absorbent pad to form quality control line (line C). The sprayed nitrocellulose membrane was dried at 25° C. for 2 h and stored in a dry place at room temperature for later use.

### 3) Assembly of Test Strip

The nitrocellulose membrane, sample pad, binding pad as well as the absorbent pad were adhered to backing plate in sequence with an overlap of 2 mm between two adjacent pads. After assembly, it was cut into 4 mm wide test strip and stored in a dry place away from light.

### Embodiment 3

### Affinity Comparison Test:

The antibody as well as nucleic acid aptamer were each modified with FITC fluorescence and incubated with Listeria monocytogenes for 1 hour. The mean fluorescence intensity was then measured using FACSCalibur flow cytometer. In the present experiment, the antibody used is the Anti-Listeria Antibody, genus specific, FITC-labeled (produced by KPL, USA and purchased from Shanghai Jinpan Biotechnology Co., Ltd.). Nucleic acid aptamer used is Listeria monocytogenes capture aptamer.

The test results are shown in FIG. 5:

The mean fluorescence intensity of antibody binding is 42.4 (a.u.), and mean fluorescence intensity of nucleic acid aptamer binding is 152 (a.u.). It can be seen that the affinity of the nucleic acid aptamer selected in the present invention is 3.6 times that of the antibody, which shows that nucleic acid aptamer has higher sensitivity and is more advantageous in preparing test strip.

The above are only preferred embodiments of the present invention, and are not intended to limit the present invention in other forms. Any skilled person familiar with the art may use the technical content disclosed above to make changes or modifications to the equivalent embodiments with equivalent changes. Any simple modifications, equivalent changes and modifications made to above embodiments based on technical essence of the present invention without departing from content of technical solution of the present invention still fall within protection scope of technical solution of the present invention.

What is claimed is:

1. A nucleic acid aptamer test strip, comprising a sample pad, a binding pad, a nitrocellulose membrane, a absorbent pad and a backing plate, wherein the binding pad contains one or more capture aptamers, wherein the nitrocellulose membrane contains one or more detection lines and one quality control line, wherein the detection line contains detection aptamer, wherein the quality control line contains quality control aptamer, and wherein the capture aptamer on the binding pad is modified onto silver-core gold-shell nanoparticles.

2. The nucleic acid aptamer test strip according to claim 1, wherein the capture aptamer is selected from the sequence shown in SEQ ID NO:1, SEQ ID NO:2 or SEQ ID NO:3, wherein the detection aptamer is selected from the sequence shown in SEQ ID NO:4, SEQ ID NO:5 or SEQ ID NO:6, and wherein the quality control aptamer is selected from the sequence shown in SEQ ID NO:7.

3. The nucleic acid aptamer test strip according to claim 2, wherein the detection aptamer can be modified with biotin and streptavidin, and wherein the quality control aptamer can be modified with biotin.

4. A method for preparing the nucleic acid aptamer test strip according to claim 1, comprising the following steps:
cutting glass cellulose membrane into strip, soaking the strip in Tris-HCl buffer solution containing NaCl, Tween-20, bovine serum albumin, sucrose as well as Triton X-100, and drying the strip to prepare sample pad as well as binding pad; spraying silver-core gold-shell nanoparticles modified with capture aptamer on binding pad, and drying strip for later use; spraying detection aptamer on nitrocellulose membrane to form detection line; drawing line on the side of quality control aptamer close to the absorbent pad to form quality control line; drying the sprayed nitrocellulose membrane for later use; sequentially adhering nitrocellulose membrane, sample pad, binding pad as well as absorbent pad to lining plate with an overlap of 2 mm between two adjacent pads, cutting the test strip into 4 mm wide strip after assembly, and storing the test strip in a dry place away from light.

5. The preparation method according to claim 4, wherein the silver-core gold-shell nanoparticles modified with capture aptamer are prepared by the following method:
adding capture aptamer to the TCEP solution for activation; placing the activated capture aptamer in silver-core gold-shell nanoparticles solution for coupling reaction; dropping NaCl solution into reaction system, mixing thoroughly, and aging at room temperature; centrifuging reaction solution, discarding supernatant, and then washing to obtain silver-core gold-shell nanoparticles modified with the capture aptamer after the reaction is completed.

6. The preparation method according to claim 5, wherein silver-core gold-shell nanoparticles are prepared by the following method:
heating water to boiling, then adding silver nitrate solution as well as trisodium citrate solution in sequence, then stirring and reacting to generate the silver nanoparticles; adding the hydroxylamine hydrochloride solution and the chloroauric acid solution to the silver nanoparticle solution, stirring and reacting, and after reaction is completed, silver-core gold-shell nanoparticles are generated.

7. An application of the nucleic acid aptamer test strip according to claim 1 in the detection of the pathogen for non-diagnostic purposes.

8. The application according to claim 7, wherein pathogen is selected from one or more of Listeria monocytogenes, Staphylococcus aureus and Escherichia coli O157:H7.
